(19) United States
(12) Patent Application Publication
(10) Pub. No.: US 2022/0062890 A1
(43) Pub. Date: Mar. 3, 2022

(54) SWAB DEVICE WITH EMBEDDED TEST ELEMENT

(71) Applicant: Daniel Larkin, St. Paul, MN (US)

(72) Inventor: Daniel Larkin, St. Paul, MN (US)

(21) Appl. No.: 17/465,498

(22) Filed: Sep. 2, 2021

Related U.S. Application Data
(60) Provisional application No. 63/074,219, filed on Sep. 3, 2020.

(51) Int. Cl. B01L 3/00; A61B 10/00; G01N 33/558; G01N 33/543; G01N 33/538

(52) U.S. Cl. CPC B01L 3/5029; A61B 10/0051; G01N 33/558; G01N 33/54388; B01L 2200/10; A61B 2010/0074; B01L 2400/0457; B01L 2300/069; B01L 2200/14; G01N 33/538

(57) ABSTRACT

A swab device, according to an example, includes a handle shaft, and a collection element positioned at a distal end of the handle shaft to collect a biological sample. The swab device includes an embedded test element to perform a test on the collected sample and provide an indication of a result of the test.

### CROSS-REFERENCE TO RELATED APPLICATION

This Non-Provisional patent application claims the benefit of the filing date of U.S. Provisional Patent Application Ser. No. 63/074,219 filed Sep. 3, 2020, entitled “SWAB WITH EMBEDDED TEST ELEMENT”, which is hereby incorporated by reference herein

### BACKGROUND

There is a need for improved sample testing techniques. For these and other reasons, a need exists for the present invention.

### SUMMARY

Some examples are directed to a swab device, which includes a handle shaft, and a collection element positioned at a distal end of the handle shaft to collect a biological sample. The swab device includes an embedded test element to perform a test on the collected sample and provide an indication of a result of the test.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a diagram illustrating a swab device with an embedded test element positioned in a collection vial according to an example.

FIG. 2 is diagram illustrating the swab device shown in FIG. 1 with zoomed-in views of the collection element of the swab device according to examples.

FIG. 3 is a diagram illustrating a swab device with an embedded test element embedded in the handle shaft, with the swab device positioned in a collection vial according to an example.

FIG. 4 is diagram illustrating the swab device shown in FIG. 3 with a zoomed-in view of a distal portion of the swab according to an example.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Some examples are directed to a swab device that includes an embedded test element for assay. In an example, the test element includes an enzyme-linked immunosorbent assay (ELISA) test element. In another example, the test element includes a lateral flow assay (LFA) test element. Some examples of the swab device are ideal for walk away, in office, or home use.

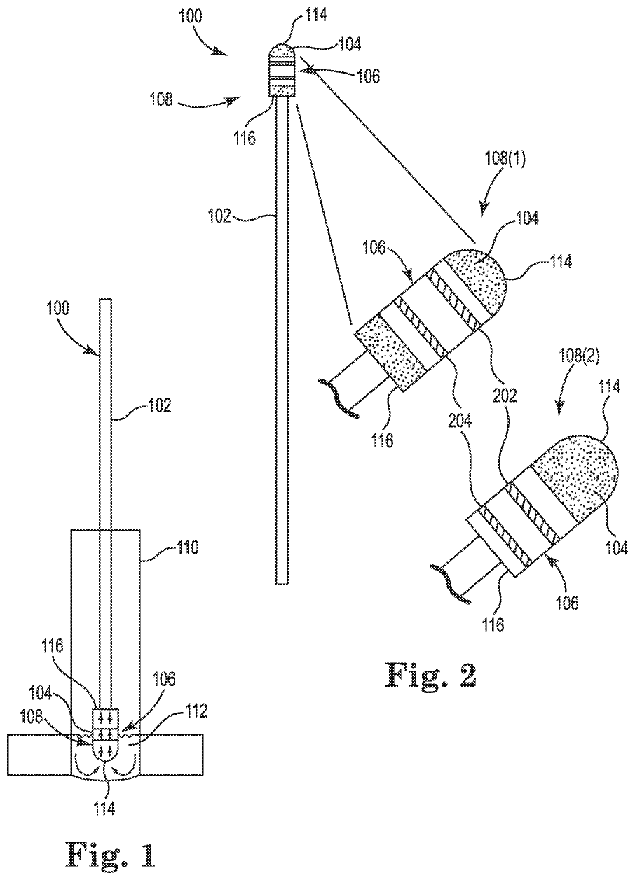

FIG. 1 is a diagram illustrating a swab device 100 with an embedded test element 106 positioned in a collection vial 110 according to an example. The swab device 100 includes a handle shaft 102 and a collection element 104. The collection element 104 is positioned at a distal end portion 108 of the handle shaft 102 and may include an absorbent material. The collection element 104 of the swab device 100 may be used to collect a biological sample, such as saliva or nasal secretions, a blood sample, an oropharyngeal sample, a penile sample, a rectal sample, a vaginal discharge sample, or any other sample. The collection element 104 of the swab device 100 may include nitrocellulose and/or other absorptive substances.

After a sample is collected, the distal end portion 108 of the swab device 100 is placed in a collection vial 110 and an assay solution 112 is added to the collection vial 110, as shown in FIG. 1. The assay solution 112 may be prepackaged in a plastic tube to add to the collection vial 110. The solution 112 is wicked up the collection element 104 of the swab device 100 from a distal end 114 of the collection element 104 toward a proximal end 116 of the collection element 104 and passes through the test element 106.

FIG. 2 is diagram illustrating the swab device 100 shown in FIG. 1 with zoomed-in views of the collection element of the swab device 100 according to examples. One zoomed-in view shows an example 108(1) of the distal end portion 108 of the swab device 100. Another zoomed-in view shows an example 108(2) of the distal end portion 108 of the swab device 100. For example 108(1), the test element 106 is positioned near a center of the collection element 104 between the distal end 114 and the proximal end 116 of the collection element 104, and includes absorbent swab material above the test element 106 near the distal end 114 and below the test element 106 near the proximal end 116.

For example 108(2), the test element 106 is positioned at the proximal end 116 of the collection element 104, and includes absorbent swab material above the test element 106 near the distal end 114, and does not include any absorbent swab material below the test element 106 near the proximal end 116.

In an example, the collection element 104 of the swab device 100 includes the test element 106. The test element 106 may include a control line element 204 and a test line element 202. After a set time period for evaluation, the control line element 204 and the test line element 202 provide an indication of a result of the test.

In an example, the test element 106 relies on gold nanoparticles to indicate the presence of a target analyte. Fluid to be tested passes from the distal end 114 of the collection element 104 of the swab device 100 and through the test element 106 toward the proximal end 116 of the collection element 104, and may also pass up into the handle shaft 102. The test line element 202 contains antibodies for the target analyte, and binds the target analyte. Excess antibodies that do not attach to the target analyte at the test line element 202 can bind specific antigens or secondary antibodies at the control line element 204. The control line element 204 is used to verify proper sample flow through the test element 106 and that the test was functional and valid.

If the target analyte is present in the sample, gold nanoparticles at the control line element 204 and test line element 202 provide a red color indication. A red line appearing at the test line element 202 indicates a positive test result (i.e., the sample includes the target analyte). A red line appearing at the control line element 204 indicates a valid test. If neither of the test line element 202 and the control line element 204 light up in a red color, this indicates a negative test result. If only the control line element 204 lights up in a red color and the test line element 202 does not light up in a red color, this indicates a negative test result. The intensity of the red lines may vary based on the amount of target analyte in the sample. A thermal contrast reader may be used in conjunction with the swab device 100 to improve the sensitivity of the test. For example, the test line element 202 may appear to indicate a negative test result to a human eye, but the thermal contrast reader may be used to read the thermal signal from the test line element 202 (and the control line element 204) to provide greater sensitivity, and may indicate that the test result is actually positive.

The test element 106 in the collection element 104 of the swab device 100 may include a continuous test strip. The test element 106 in the collection element 104 of the swab device 100 may include two separate embedded test discs (e.g., a first disc for test line element 202, and a second disc for control line element 204) sandwiched in absorbent collection swab material. In some examples, the test line element 202 and control line element 204 may be embedded in the swab material of the collection element 104. In other examples, the test line element 202 and control line element 204 may be embedded in or on the handle shaft 102 of the swab device 100.

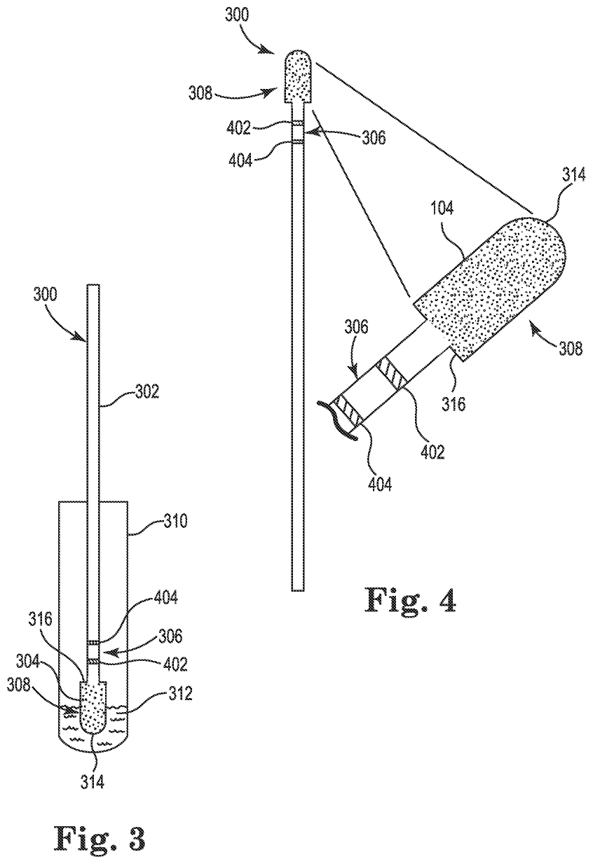

FIG. 3 is a diagram illustrating a swab device 300 with an embedded test element 306 embedded in the handle shaft 302, with the swab device 300 positioned in a collection vial 310 according to an example. FIG. 4 is diagram illustrating the swab device 300 shown in FIG. 3 with a zoomed-in view of a distal portion 308 of the swab device 300 according to an example. The swab device 300 shown in FIGS. 3 and 4 may be used to test a sample in the same or similar manner as described above with respect to FIGS. 1 and 2. The swab device 300 includes a handle shaft 302 and a collection element 304. The collection element 304 is positioned at a distal end of the handle shaft 302 and may include an absorbent material. The collection element 304 of the swab device 300 may be used to collect a biological sample, such as saliva or nasal secretions, a blood sample, an oropharyngeal sample, a penile sample, a rectal sample, a vaginal discharge sample, or any other sample. The collection element 304 of the swab device 300 may include nitrocellulose and/or other absorptive substances. After a sample is collected, the distal end portion 308 of the swab device 300 is placed in a collection vial 310 and an assay solution 312 is added to the collection vial 310, as shown in FIG. 3. The assay solution 312 may be prepackaged in a plastic tube to add to the collection vial 310. The solution 312 is wicked up the collection element 304 of the swab device 300 from a distal end 314 of the collection element 304 toward a proximal end 316 of the collection element 304 and passes through the test element 306.

The swab device 300 includes a test element 306 positioned on the handle shaft 302 near a distal end of the handle shaft 302 and proximal to the proximal end 316 of the collection element 304. The test element 306 may include a control line element 404 and a test line element 402. The test line element 402 may be positioned distal to the control line element 404. After a set time period for evaluation, the control line element 404 and the test line element 402 provide an indication of a result of the test.

In an example, the test element 306 relies on gold nanoparticles to indicate the presence of a target analyte. Fluid to be tested passes from the distal end 314 of the collection element 304 of the swab device 300 toward the proximal end 316 of the collection element 304, and also passes up into the handle shaft 302 and through the test element 306. The test line element 402 contains antibodies for the target analyte, and binds the target analyte. Excess antibodies that do not attach to the target analyte at the test line element 402 can bind specific antigens or secondary antibodies at the control line element 404. The control line element 404 is used to verify proper sample flow through the test element 306 and that the test was functional and valid.

If the target analyte is present in the sample, gold nanoparticles at the control line element 404 and test line element 402 provide a red color indication. A red line appearing at the test line element 402 indicates a positive test result (i.e., the sample includes the target analyte). A red line appearing at the control line element 404 indicates a valid test. If neither of the test line element 402 and the control line element 404 light up in a red color, this indicates a negative test result. If only the control line element 404 lights up in a red color and the test line element 402 does not light up in a red color, this indicates a negative test result. The intensity of the red lines may vary based on the amount of target analyte in the sample. A thermal contrast reader may be used in conjunction with the swab device 300 to improve the sensitivity of the test. For example, the test line element 402 may appear to indicate a negative test result to a human eye, but the thermal contrast reader may be used to read the thermal signal from the test line element 402 (and the control line element 404) to provide greater sensitivity, and may indicate that the test result is actually positive.

The test element 306 on the handle shaft 302 of the swab device 300 may include a continuous test strip. The test element 306 on the handle shaft 302 of the swab device 300 may include two separate embedded test discs sandwiched in absorbent collection swab material.

An example is directed to a swab device, which includes a handle shaft, and a collection element positioned at a distal end of the handle shaft to collect a biological sample. The swab device includes an embedded test element to perform a test on the collected sample and provide an indication of a result of the test.

The collection element may include an absorbent swab material. The embedded test element may be embedded in the collection element. The embedded test element may be positioned near a center of the collection element, and the collection element may include a first portion of the absorbent swab material proximal to the embedded test element and a second portion of the absorbent swab material distal to the embedded test element. The embedded test element may be positioned at a proximal end of the collection element, and the absorbent swab material of the collection element may be positioned distal to the embedded test element.

The embedded test element may be embedded in the handle shaft. The embedded test element may be positioned adjacent to a proximal end of the collection element.

The embedded test element may include a test line element and a control line element. The test line element may contain antibodies for a target analyte and provide an indication of whether the target analyte is present in the biological sample, and the control line element may indicate whether the test is valid. The embedded test element may include a continuous test strip. The embedded test element may include two separate embedded discs.

An assay solution may be applied to the collection element as part of the test. The embedded test element may include an enzyme-linked immunosorbent assay (ELISA) test element. The embedded test element may include a lateral flow assay (LFA) test element.

Another example is directed to a swab device, which includes a handle shaft, and a collection element with absorbent swab material positioned at a distal end of the handle shaft to collect a biological sample. The swab device includes a test element embedded in one of the handle shaft or the collection element to perform a test on the collected sample and provide an indication of a result of the test. The test element includes a test line element to provide an indication of whether a target analyte is present in the biological sample and a control line element to indicate whether the test is valid.

The test element may be embedded in the collection element. The test element may be embedded in the handle shaft.

Another example is directed to a method, which includes collecting a biological sample with a collection element of a swab device, wherein the collection element comprises absorbent swab material and is positioned at a distal end of a handle shaft of the swab device. The method includes performing a test on the collected sample with a test element embedded in the swab device, wherein the test element provides an indication of a result of the test.

Performing the test in the method may include applying an assay solution to the collection element after the biological sample is collected, and the test element may include a test line element containing antibodies for a target analyte and that provides an indication of whether the target analyte is present in the biological sample, and the test element may include a control line element to indicate whether the test is valid. The method may include using a thermal contrast reader to read a thermal signature from the test element.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A swab device, comprising:
a handle shaft;
a collection element positioned at a distal end of the handle shaft to collect a biological sample; and
an embedded test element to perform a test on the collected sample and provide an indication of a result of the test.

2. The swab device of claim 1, wherein the collection element comprises an absorbent swab material.

3. The swab device of claim 2, wherein the embedded test element is embedded in the collection element.

4. The swab device of claim 3, wherein the embedded test element is positioned near a center of the collection element, and wherein the collection element includes a first portion of the absorbent swab material proximal to the embedded test element and a second portion of the absorbent swab material distal to the embedded test element.

5. The swab device of claim 4, wherein the embedded test element is positioned at a proximal end of the collection element, and wherein the absorbent swab material of the collection element is positioned distal to the embedded test element.

6. The swab device of claim 1, wherein the embedded test element is embedded in the handle shaft.

7. The swab device of claim 6, wherein the embedded test element is positioned adjacent to a proximal end of the collection element.

8. The swab device of claim 1, wherein the embedded test element includes a test line element and a control line element.

9. The swab device of claim 8, wherein the test line element contains antibodies for a target analyte and provides an indication of whether the target analyte is present in the biological sample, and wherein the control line element indicates whether the test is valid.

10. The swab device of claim 8, wherein the embedded test element comprises a continuous test strip.

11. The swab device of claim 8, wherein the embedded test element comprises two separate embedded discs.

12. The swab device of claim 1, wherein an assay solution is applied to the collection element as part of the test.

13. The swab device of claim 1, wherein the embedded test element comprises an enzyme-linked immunosorbent assay (ELISA) test element.

14. The swab device of claim 1, wherein the embedded test element includes a lateral flow assay (LFA) test element.

15. A swab device, comprising:
a handle shaft;
a collection element with absorbent swab material positioned at a distal end of the handle shaft to collect a biological sample; and
a test element embedded in one of the handle shaft or the collection element to perform a test on the collected sample and provide an indication of a result of the test, wherein the test element includes a test line element to provide an indication of whether a target analyte is present in the biological sample and a control line element to indicate whether the test is valid.

16. The swab device of claim 15, wherein the test element is embedded in the collection element.

17. The swab device of claim 15, wherein the test element is embedded in the handle shaft.

18. A method, comprising:
collecting a biological sample with a collection element of a swab device, wherein the collection element comprises absorbent swab material and is positioned at a distal end of a handle shaft of the swab device; and
performing a test on the collected sample with a test element embedded in the swab device, wherein the test element provides an indication of a result of the test.

19. The method of claim 18, wherein performing the test comprises:
applying an assay solution to the collection element after the biological sample is collected, and wherein the test element includes a test line element containing antibodies for a target analyte and that provides an indication of whether the target analyte is present in the biological sample, and wherein the test element includes a control line element to indicate whether the test is valid.

20. The method of claim 18, and further comprising:
using a thermal contrast reader to read a thermal signature from the test element.
